(19) United States
(12) Patent Application Publication
(10) Pub. No.: US 2017/0266396 A1
(43) Pub. Date: Sep. 21, 2017

(54) NASAL SPRAY APPARATUS

(71) Applicants: JOSEPH FELDMAN, TEL AVIV (IL); NITZAN PRIMOR, TEL AVIV (IL)

(72) Inventors: JOSEPH FELDMAN, TEL AVIV (IL); NITZAN PRIMOR, TEL AVIV (IL)

(21) Appl. No.: 15/464,388

(22) Filed: Mar. 21, 2017

Related U.S. Application Data
(60) Provisional application No. 62/310,824, filed on Mar. 21, 2016.

(51) Int. Cl. A61M 11/02; A61N 5/06; A61M 11/00; A61M 31/00

(52) U.S. Cl. CPC A61M 11/02; A61M 31/00; A61N 5/0603; A61M 11/006; A61N 2005/0607; A61N 2005/0651

(57) ABSTRACT

A cap for a nasal spray apparatus, the cap including a spray dispensing mechanism and further including a base sealingly mountable on a container of liquid substance to be dispensed; an intermediate section arranged for reciprocating movement relative to the base the intermediate section including the spray dispensing mechanism and having a spray dispensing passage; a cover-section having a depressible flexible portion, coupled to the intermediate-section; and a Light Emitting Diode (LED) connected to a power source through an electric circuit, the LED mounted adjacent the spray dispensing passage so as to be inserted into a nostril therewith; wherein the depressible flexible portion is arranged and configured to cause closure of the electric circuit for activating the LED and to actuate the spray dispensing mechanism, to provide both illumination and spray of liquid substance into the nostril.

### RELATED APPLICATIONS

This application claims priority from U.S. provisional patent application Ser. No. 62/310,824, filed 21 Mar. 2016.

### FIELD OF THE INVENTION

The present invention relates to nasal spray apparatus.

### BACKGROUND OF THE INVENTION

The present invention concerns devices for administrating medicated (or non-medicated) fluid substances, in the form of droplets or aerosol spray, to the nostrils of patients suffering from nasal congestion, typically due to allergic diseases (“rhinitis”). Clinical symptoms of rhinitis, such as a runny nose, an itchy nose, post nasal drainage of mucus and, in extreme cases, congested secondary air passages, may be attributed to various etiologies. The common etiologies are viral infection, such as infectious rhino sinusitis. Other etiologies include allergic, perennial, or seasonal rhinitis, also known as “hay fever”, non-allergic vasomotor rhinitis, eosinophyllic rhinitis and nasal polyps.

Existing methods of treatment of the above mentioned rhinitis symptoms include systematic use of medications, such as antihistamines and decongestants, or local treatment with steroid spray, D.S.C.G. or local decongestants. There have been also attempts to treat rhinitis locally by applying a “fog stream”, i.e. a stream of water at a temperature of approximately 42° C. These medications may be taken orally or may be administered directly to the tissue of the nostrils by means of a nasal spray.

The majority of the commercially available atomizers or aerosol spray devices comprise a pressurized propellant gas vessel containing the active substance in a liquid form (e.g. antihistamine, sea water), with a suitable shaped cap so that by pressing thereon, a valve is opened and the spray is ejected into the user's nostril for as long as the cover is compressed. Other models have a pumping mechanism rather than compressed gas. According to these models each time the cover is pressed a pre-selected amount of the substance is dispensed.

It is appreciated that none of the existing treatments described above, nor any combinations thereof, completely relieve rhinitis related symptoms. Therefore, a large population is helplessly exposed to the irritating discomforts of rhinitis symptoms.

As an alternative means of treatment of rhinitis, a line of nasal congestion treatment devices have been developed based on nostril illumination of the type disclosed in U.S. Pat. No. 5,683,436 (Nov. 4, 1997—Mendes, et al.). This patent provides a method and apparatus for therapeutic illumination which are particularly suited for treatment of rhinitis and for treatment of various nasal conditions. As therein described, the operation of these biostimulative illumination apparatus includes the use of Light Emitting Diodes (LED) configured to radiate non-coherent red light at a preferred wavelength of 660 nm into the nostril.

Accordingly, there is a long felt need for a spraying device that is capable of administering the medicated (or non-medicated) fluid while illuminating the area that is being sprayed, and it would be desirable to have a spraying device that permits the performing of these two operations by a single action of the user.

### SUMMARY OF THE INVENTION

The present invention combines the two therapeutic methodologies into a unified, self-contained nasal spray apparatus. The single nasal spray apparatus activates an LED to provide biostimulative illumination and, simultaneously, administers the active fluid. According to embodiments of the invention, both operations are initiated by depressing the nozzle cap.

There is provided according to the present invention a cap for a nasal spray apparatus, the cap including a spray dispensing mechanism and further including a base sealingly mountable on a container of liquid substance to be dispensed; an intermediate section arranged for reciprocating movement relative to the base the intermediate section including the spray dispensing mechanism and having a spray dispensing passage; a cover-section having a depressible flexible portion, coupled to the intermediate-section; and a Light Emitting Diode (LED) connected to a power source through an electric circuit, the LED mounted adjacent the spray dispensing passage so as to be inserted into a nostril therewith; wherein the depressible flexible portion is arranged and configured to cause closure of the electric circuit for activating the LED and to actuate the spray dispensing mechanism, to provide both illumination and spray of liquid substance into the nostril.

There is provided according to a general aspect of the invention is an atomizer apparatus for treating nasal congestion and the like conditions of a patient. The apparatus comprises a vessel containing a therapeutic fluid intended to be administered in droplet (spray) form into a nostril of the patient. According to some embodiments of the invention, valve means are provided for controlling the delivery of the fluid by a push-button nozzle cap member mounted on the vessel. By pressing the cap member, communication is established between the valve means and spray dispensing passage in the cap configured to be inserted into the nostril for moistening the nostril by the fluid. A Light Emitting Diode (LED) is provided, adjacent the spray dispensing passage, so as to be inserted into the nostril therewith. Further provided are an electric power source connectable via switching means to the LED and means for selectively actuating the switching means by the patient.

### BRIEF DESCRIPTION OF THE DRAWINGS

The objects, constructional features and advantages of the present invention will be more readily understood in the light of the ensuing description of a preferred embodiment thereof, given by way of example only, with reference to the accompanying drawings, wherein:

FIG. 1 is a general side-view of nasal spray apparatus constructed and operative according to preferred embodiments of the invention;

FIG. 2 is a perspective exploded view of a cap for the nasal spray apparatus of FIG. 1;

FIG. 3A is a side-view of nozzle cap member of FIG. 2;

FIG. 3B is a cross-section taken along A-A of FIG. 3A;

FIG. 3C is a cross-section taken along B-B of FIG. 3A;

FIG. 4A is a top-view of the nozzle cap member of FIG. 3A;

FIG. 4B is a cross-section taken along C-C of FIG. 4A; and,

FIG. 4C is a cross-section taken along D-D of FIG. 4A.

### DETAILED DESCRIPTION OF THE INVENTION

The present invention relates to a cap for a nasal spraying device that is capable of administering a medicated (or non-medicated) fluid, in spray form, while illuminating the area that is being sprayed. In particular, the nasal spraying device permits performing these two operations substantially simultaneously, or one immediately after the other, by a single action of the user with a single device. In this way, more effective treatment for rhinitis and the like can be provided by a single device in a single operation.

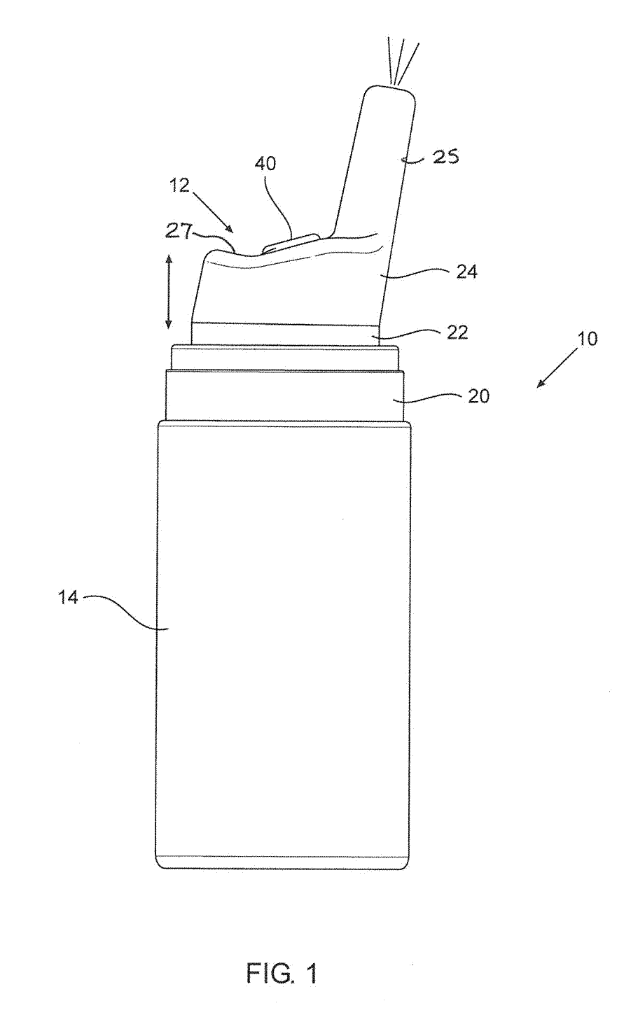

FIG. 1 illustrates a nasal spray apparatus according to a preferred embodiment of the present invention, generally designated 10. The embodiment of nasal spray apparatus 10 utilizes an aerosol delivery mechanism. A nozzle cap member 12, in its assembled state, is sealingly mounted onto a container 14, which can hold a propellant gas as well as a liquid substance for spraying. Typically in aerosol mechanisms, a long plastic tube rims from the bottom of the container up to a valve system (not shown) at the top of the container. The valve can have a narrow channel running from an inlet near the bottom of a head piece to a small nozzle at the top. A spring pushes the head piece up, so the channel inlet is blocked by a tight seal. When the head piece is pushed down, the inlet slides below the seal, opening a passage from the inside of the container to the spray passage. The high-pressure propellant gas drives the liquid product up the plastic tube, through the spray passage and out through a nozzle or spray orifice. The narrow nozzle serves to atomize the flowing liquid, breaking it up into tiny drops, which form a fine spray.

In this embodiment of the invention, depressing the cap member towards the container will cause dispensing of liquid from the container and into the nostril of the user. A flexible portion, possibly including a depressible actuator button 40, is provided in cap member 12, the construction and function of which will be described in greater detail below. Essentially, pressing down on the flexible portion opens the valve and allows pressurized contents from the container to be sprayed out through the spray orifice. The illumination effect is improved when the spray drops are relatively small in size.

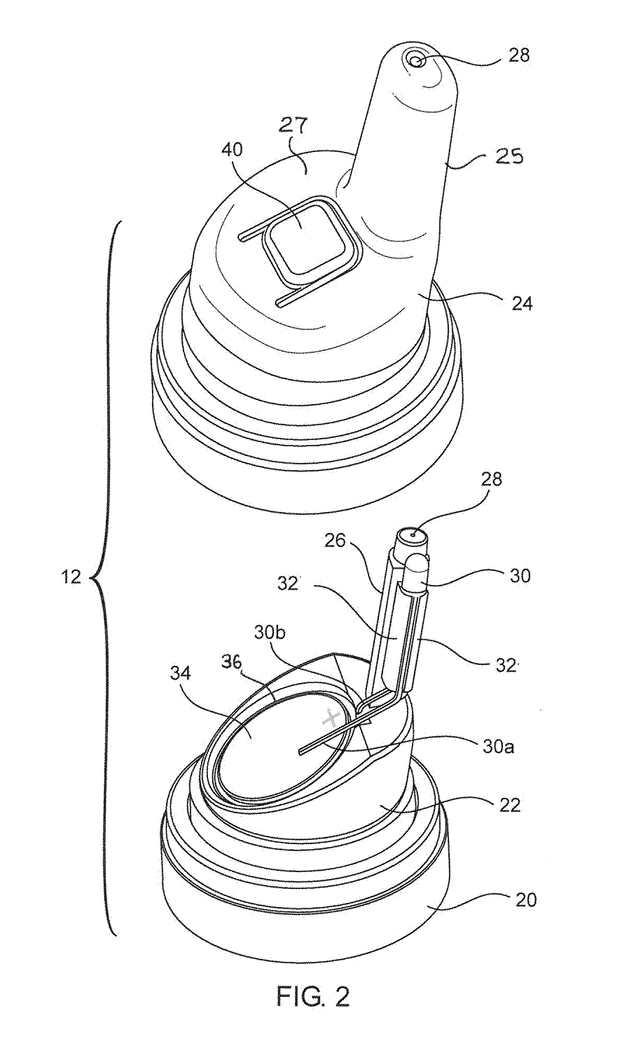

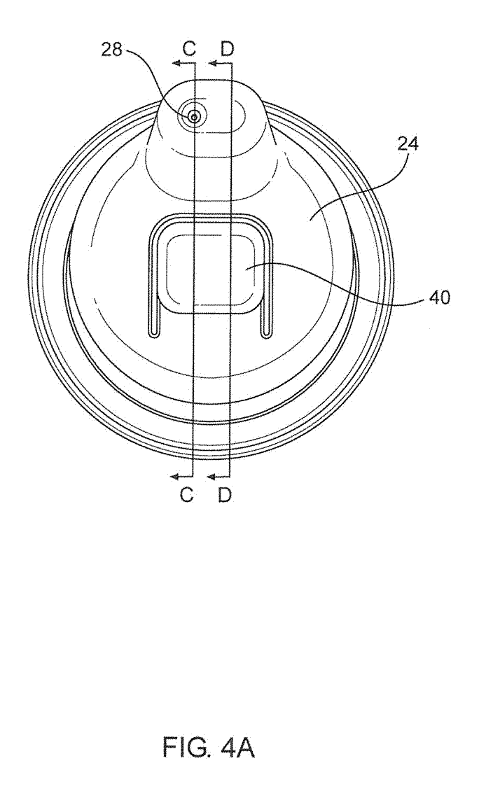

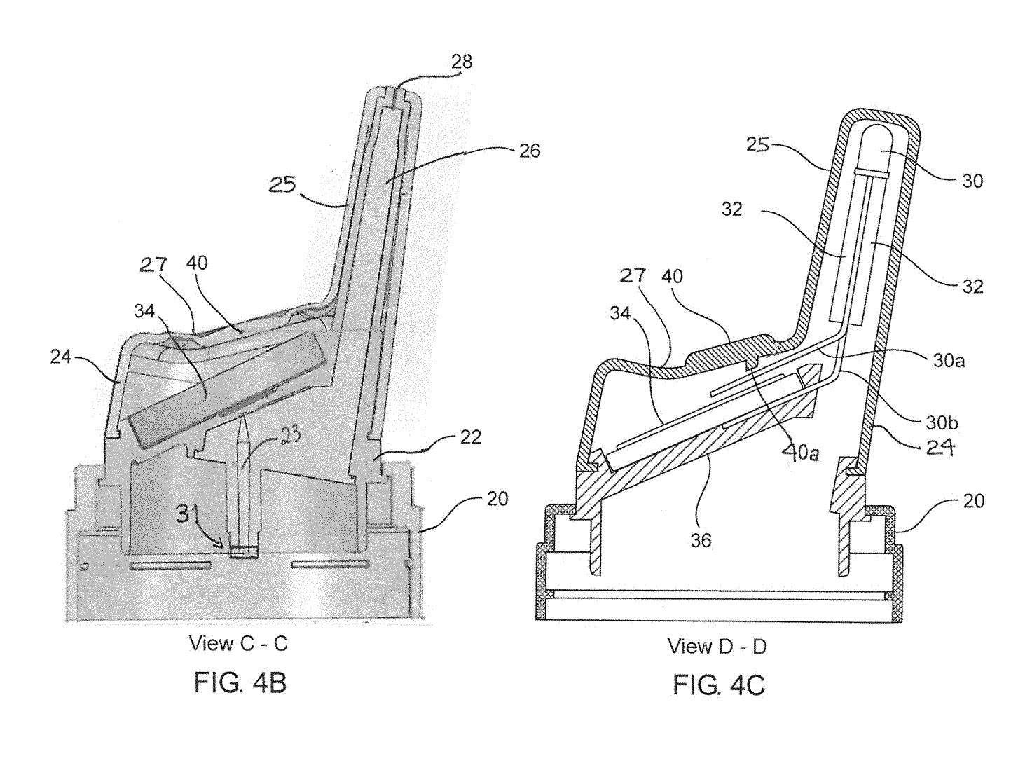

With further reference to FIGS. 2, 4B and 4C, the cap member 12 according to the illustrated embodiment is composed of three assembled-together sections: A base-section 20, which is coupled to the container 14; an intermediate-section 22 including a spray dispensing mechanism, the intermediate section configured and adapted for reciprocating movement (to slide up and down) relative to the base-section to operate a gas/fluid valve, generally indicated 23, of the container by any known means (not shown); and a cover-section 24, including a dispensing nozzle 25, press-fitted (or otherwise assembled) to the intermediate-section 22. The spring in the aerosol valve causes intermediate-section 22 to return to its usual position.

In more detail, the intermediate-section 22 is formed with a tubular spray passage 26 which is seated inside dispensing nozzle 25, and which has a dispensing orifice 28. When the apparatus is operated, the spray liquid passes from the container, through the spray passage 26 and is dispensed from orifice 28. The intermediate-section 22 further accommodates a Light Emitting Diode (LED) 30 mounted adjacent spray passage 26, preferably juxtaposed to the spray orifice 28. Preferably, LED 30 emits non-coherent light radiation having a narrow bandwidth centered at a wavelength suitable for rhinitis treatment, typically 660 nm.

Preferably, the liquid to be dispensed includes a photosensitizer, such as methylene blue. The photosensitizer increases the sensitivity of bacteria and other organisms or substances in the nostril to electromagnetic radiation, especially visible light, increasing the absorption of light into their cells and disrupting their biochemical balance. In this way, the effect of the light radiation is multiplied and substantially improves the effect of the treatment.

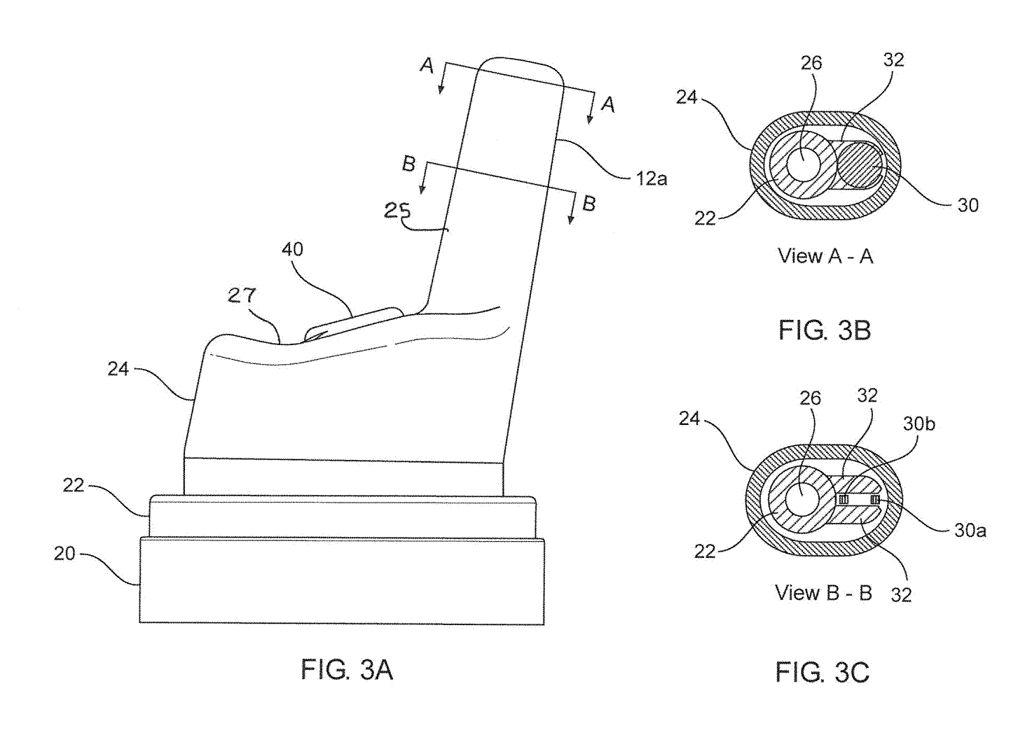

The LED is mounted adjacent the spray dispensing passage so as to be inserted into a nostril together therewith. LED 30 is mounted in a support 32 formed of insulating material including two channels or bores which holds and separates the terminals, anode 30a and cathode 30b, of the LED. This structure can be seen most clearly in FIGS. 3A, 3B and 3C. As can be seen in FIG. 3C, the tubular spray passage 26 is affixed to or integrally formed with the support 32, through which pass terminals 30a and 30b of the LED. In this embodiment, as shown in FIG. 3B, the LED 30 is juxtaposed with the upper end of spray passage 26, preferably adjacent the spray orifice 28. LED 30 is connected to a power source through an electric circuit, and the depressible flexible portion is arranged and configured to cause the electric circuit to close to activate the LED and also to actuate the spray dispensing mechanism, to provide both illumination and spray of liquid substance into the nozzle. Preferably, the LED illuminates the spray as well as the nostril.

A power source, here shown as a coin-type battery 34, is seated within a battery compartment 36. LED cathode 30b is disposed in battery compartment 36 under battery 34 and contacts the bottom side of the battery 34, the negative pole of the battery, while the anode 30a is disposed above—but is flexibly kept a small distance away from—the upper side of the battery, the positive pole of the battery. It will be appreciated that battery 34 can be fitted in battery compartment 36 upside down, i.e. its negative pole is facing upward and positive pole is facing downward while the terminals of LED 30 are switched wherein anode 30a is disposed in battery compartment 36 under battery 34 and makes contact with the positive pole of the battery 34 and cathode 30b is displace above the battery a small distance away from it.

The cover-section 24 is placed over the intermediate-section 22. It comprises a flexible upper wall 27 which can be used to actuate the spray apparatus. Upper wall 27 is sufficiently flexible to be depressed and engage a terminal of the LED so as to cause the terminal to contact the battery and light up the LED. Preferably, upper wall 27 can continue to press on the intermediate section 22 until it slides relative to base 20 and actuates the aerosol dispenser.

According to some embodiments of the invention, a push-button 40 is provided protruding from the upper wall 27 for ease of actuation. A further protrusion 40a (best seen in FIGS. 4B and 4C) can be formed at the bottom side of button 40 disposed so that by pressing the button down, protrusion 40a depresses LED anode 30a until it contacts the upper side of the battery 34, closing the electric circuit which includes the battery and the LED, and lighting the LED. It will be appreciated that push-button 40 can be integrally formed with upper wall 27 of section 24 and/or protrusion 40a can be integrally formed with upper wall 27. Continued pressing of push-button 40 causes intermediate section 22 to slide relative to base 20, causing a valve actuator 23 to open the valve 31, as in conventional aerosol mechanisms.

In the light of the foregoing description it will be clearly understood that the operation of the apparatus according to the present invention may be carried out in either manner By merely pressing the button 40—the LED will become activated and a biostimulative treatment can be achieved for as long as recommended; in addition, by further, more forceful pressing of button 40 section 24 will move section 22 downward and spraying of liquid from the container 14 will take place as in the conventional nasal treatment devices, while LED 30 is activated according to the present invention. In this way, both therapeutic treatments can be provided at the same time to the nostril of a user.

Those skilled in the art to which this invention pertains will readily appreciate that numerous changes, variations and modifications can be effectuated without departing from the true spirit and scope of the invention as defined in and by the appended claims. For example, a manually operated switch can be provided for closing the circuit and lighting the LED, instead of the push-button described above. The switch can be connected on one side to one of the terminals of the LED and the compatible pole of the battery, which can be disposed anywhere in the cap. The other terminal of the LED is connected to the other pole of the battery. For operation, the user actuates the switch which closes the circuit and lights the LED. Thereafter, the user can continue to press the switch mounted on the flexible intermediate portion and the pressure would open the valve of the aerosol and release the spray.

Instead of aerosol, the present invention alternatively can utilize any known mechanism of spray delivery, such as an atomizer operating under the Venturi effect, or a nasal spray pump mechanism. In the latter case, pressing down on the push-button or flexible intermediate portion would cause contact of the terminals with the battery to light the LED and continued pressure would produce a spray of substance out of nozzle by means of a positive displacement pump that acts directly on the fluid. As known, the pump draws liquid up a siphon tube from the bottom of the container and forces it through the nozzle or spray orifice.

In the case of a mechanism for creating spray other than the aerosol described above, the cap for the spray apparatus would also include three assembled-together sections as described above: A base-section 20, which is sealingly coupled to the liquid container; an intermediate-section 22 including a spray dispensing mechanism, the intermediate section configured and adapted for reciprocating movement (to slide up and down) relative to the base-section to release liquid from the container; and a cover-section 24, including a dispensing nozzle 25, press-fitted (or otherwise assembled) to the intermediate-section.

In more detail, the intermediate-section 22 is formed with a tubular spray passage 26 which is seated inside dispensing nozzle 25, and which has a dispensing orifice 28. When the apparatus is operated, the spray liquid passes from the container, through the spray passage 26 and is dispensed from orifice 28. The intermediate-section 22 further accommodates a Light Emitting Diode (LED) 30 mounted adjacent spray passage 26, preferably juxtaposed to the spray orifice 28 so as to be inserted into a nostril together. Preferably, LED 30 emits non-coherent light radiation having a narrow bandwidth centered at a wavelength suitable for rhinitis treatment, typically 660 nm.

LED 30 is mounted in a support 32 formed of insulating material including two channels or bores which holds and separates the terminals 30a, 30b of the LED, as described above with regard to FIGS. 3A, 3B and 3C. LED 30 is connected to a power source through an electric circuit, and the depressible flexible portion is arranged and configured to cause the electric circuit to close to activate the LED and also to actuate the spray dispensing mechanism, to provide both illumination and spray of liquid substance into the nozzle. Preferably, the LED illuminates the spray as well as the nostril.

A power source, here shown as a coin-type battery 34, is seated within a battery compartment 36. One LED terminal is disposed in battery compartment 36 under battery 34 and contacts the compatible pole of the battery 34, while the second terminal 30a is disposed above—but is flexibly kept a small distance away from—the upper side of the battery.

The cover-section 24 is placed over the intermediate-section 22. It comprises a flexible upper wall 27 which can be used to actuate the spray apparatus. Upper wall 27 is sufficiently flexible to be depressed and engage a terminal of the LED so as to cause the terminal to contact the battery and light up the LED. Preferably, upper wall 27 can continue to press on the intermediate section 22 until it slides relative to base 20 and actuates the aerosol dispenser.

If desired, a push-button 40 can be provided protruding from the upper wall 27 for ease of actuation. A further protrusion 40a (best seen in FIGS. 4B and 4C) can be formed at the bottom side of button 40 disposed so that by pressing the button down, protrusion 40a depresses LED terminal 30a until it contacts the upper side of the battery 34, closing the electric circuit which includes the battery and the LED, and lighting the LED. It will be appreciated that push-button 40 can be integrally formed with upper wall 27 of section 24 and/or protrusion 40a can be integrally formed with upper wall 27. Continued pressing of push-button 40 causes intermediate section 22 to slide relative to base 20, causing the liquid dispensing mechanism to dispense liquid from the container.

It will be appreciated that the invention is not limited to what has been described hereinabove merely by way of example. Rather, the invention is limited solely by the claims which follow.

What is claimed is:

1. A cap for a nasal spray apparatus, the cap including a spray dispensing mechanism and further comprising:
a base sealingly mountable on a container of liquid substance to be dispensed;
an intermediate section arranged for reciprocating movement relative to the base the intermediate section including the spray dispensing mechanism and having a spray dispensing passage;
a cover-section having a depressible flexible portion, coupled to the intermediate-section; and
a Light Emitting Diode (LED) connected to a power source through an electric circuit, the LED mounted adjacent the spray dispensing passage so as to be inserted into a nostril therewith;
wherein the depressible flexible portion is arranged and configured to cause closure of the electric circuit for activating the LED and to actuate the spray dispensing mechanism, to provide both illumination and spray of liquid substance into the nostril.

2. The cap according to claim 1, wherein :
the LED includes two terminals;
the power source is a battery seated in a battery compartment disposed in the cap beneath the depressible flexible portion;
wherein one terminal of the LED is disposed between the depressible flexible portion and the battery and not in contact with the battery, and the second terminal of the LED is disposed in the battery compartment beneath and in contact with the battery.

3. The cap according to claim 1, wherein the depressible flexible portion includes a protruding push-button on one side and an LED terminal engaging protrusion on the other side.

4. The cap according to claim 2, wherein the depressible flexible portion includes a protruding push-button on one side and an LED terminal engaging protrusion on the other side.

5. The cap according to claim 1, wherein the depressible flexible portion includes a manual switch for closing the electric circuit to illuminate the LED.

6. A method for dispensing a nasal treatment liquid substance, the method comprising:
dispensing a spray of liquid substance through a spray passage; and
lighting an LED mounted adjacent the spray passage simultaneously with the step of dispensing so as to illuminate the spray and an area moistened by the spray.

7. The method according to claim 6, wherein the step of lighting is performed before and during the step of dispensing.

8. The method according to claim 6, wherein the steps of dispensing and lighting are accomplished by pressing a flexible wall of a cap member against a first terminal of the LED until it contacts a compatible pole of a battery while a second terminal of the LED contacts a second pole of the battery to close an electric circuit, and continued pressing on the flexible wall causes dispensing of the spray.

9. The method according to claim 6, further comprising adding a photosensitive material to the liquid substance before the step of dispensing.

10. The method according to claim 7, further comprising adding a photosensitive material to the liquid substance before the step of dispensing.

11. The method according to claim 8, further comprising adding a photosensitive material to the liquid substance before the step of dispensing.
